(19) United States
(12) Patent Application Publication
(10) Pub. No.: US 2023/0390384 A1
(43) Pub. Date: Dec. 7, 2023

(54) RECOMBINANT COVID-19 VACCINE COMPOSITION COMPRISING LIPOPEPTIDE AND POLY (I:C) ADJUVANT, AND USE THEREOF

(71) Applicant: CHA VACCINE RESEARCH INSTITUTE CO., LTD, Seongnam-si (KR)

(72) Inventors: Jung Sun Yum, Seongnam-si (KR); Byung Cheol Ahn, Yongin-si (KR); Sookyung Jeong, Anyang-si (KR); Yoonki Heo, Suwon-si (KR); Sujin Ham, Namyangju-si (KR); Eunyoung Chun, Seongnam-si (KR)

(73) Assignee: CHA VACCINE RESEARCH INSTITUTE CO., LTD, Seongnam-si (KR)

(21) Appl. No.: 18/035,884

(22) PCT Filed: Nov. 8, 2021
(86) PCT No.: PCT/KR2021/016107

§ 371 Date: May 8, 2023

(30) Foreign Application Priority Data
Nov. 9, 2020 (KR) 10-2020-0148386

(51) Int. Cl. A61K 39/215; A61K 39/39; A61P 31/14

(52) U.S. Cl. CPC A61K 39/215; A61K 2039/55516; A61P 31/14; A61K 39/39

(57) ABSTRACT

The present invention relates to a recombinant COVID-19 vaccine composition comprising a lipopeptide and a poly(I:C) adjuvant. The vaccine composition for preventing or treating COVID-19, provided in one aspect of the present invention, can greatly induce both a humoral immune response and a cellular immune response to a recombinant COVID-19 antigen, and thus can be developed as a COVID-19 vaccine so as to be commercially and effectively usable.

### BACKGROUND OF THE INVENTION

### 1. Field of the Invention

The present invention relates to a recombinant COVID-19 vaccine composition comprising a lipopeptide and a poly(I:C) adjuvant, and a use thereof.

### 2. Description of the Related Art

COVID-19, which occurred in Wuhan, Hubei Province, China in December 2019, had spread to about 114 countries by March 2020. Accordingly, the World Health Organization (WHO) declared that COVID-19 is a global pandemic situation. Since then, by September 2020, about 35 million infected patients have occurred worldwide, of which about 1 million have died, and the number of infected patients and deaths continues to increase.

COVID-19 shows respiratory symptoms such as dry cough, sputum, and difficulty in breathing along with fever, and can cause complications such as acute dyspnea syndrome, heart failure, and arrhythmia. In this case, conservative treatment is being conducted through oxygen therapy, and administration of antiviral agents and antibiotics, but there is a problem that sufficient effect does not appear because it is not a cure for coronavirus.

COVID-19 is mainly infected by the spread of droplets caused by coughing or sneezing, and the basic reproduction number (average number of people infected by one infected person during contagious period) is 2 to 2.5, which is known to be more contagious than the flu. In addition, in the case of COVID-19, even asymptomatic infected people can spread the virus, making it difficult to effectively block the virus. Therefore, it is important to develop a vaccine that can effectively prevent COVID-19, which has no fundamental treatment and is highly contagious.

Meanwhile, coronavirus (CoV) phylogenetically refer to a virus belonging to Coronaviridae, and the subgroup Ortho Coronaviridae is classified into four genera: alpha-CoV, beta-CoV, delta-CoV, and gamma-CoV. Among them, only alpha-CoV and beta-CoV infect mammals, while delta-CoV and gamma-CoV infect birds and some mammals.

So far, there are seven types of coronavirus (HCoV) that can infect humans: HCoV-229E and HCoV-NL63 of alpha-CoV, HCoV-0C43, HCoV-HKU1, SARS-CoV and MERS-CoV of beta-CoV, and SARS-CoV-2, the causative agent of the 2019 coronavirus disease (COVID-19). HCoV-229E, HCoV-NL63, HCoV-0C43 and HCoV-HKU1 cause common colds or gastrointestinal diseases in humans, but along with SARS-CoV and MERS-CoV, SARS-CoV-2 causes severe acute respiratory infectious diseases.

SARS-CoV-2 belongs to beta-CoV in the phylogenetic tree along with SARS-CoV and MERS-CoV, but it is clearly distinct from SARS-CoV in terms of molecular phylogeny and evolved quite a long time ago from MERS-CoV, which has about half of its nucleotide sequence similarity, as shown in the evolutionary analysis of SARS-CoV, MERS-CoV and SARS-CoV-2 using the maximum likelihood method.

[Evolutionary analysis of SARS-CoV, MERS-CoV-2 and SARS-CoV-2, Maximum Likelihood]

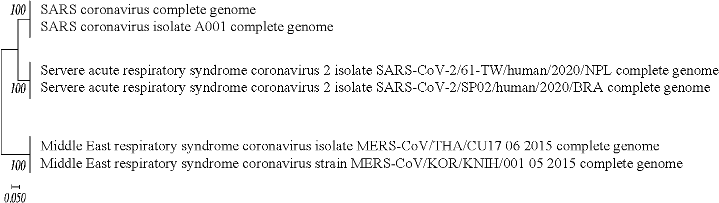

In addition, SARS-CoV-2 has some similarities in structure and pathogenicity compared to SARS-CoV, but there is a clear structural difference in the protein structure, that is, the spike protein (S), which should be considered most important for vaccine development. The presence of a furin-like cleavage site (SLLR-ST) in SARS-CoV-2 promotes priming of S protein, further increasing transmissibility of SARS-CoV-2 compared to SARS-CoV (Non-patent reference 1, Le Infezioni in Medicina, n. 2, 174-184, 2020, SARS-CoV-2, SARS-CoV, and MERS-CoV: a comparative overview).

More specifically, cleavage of the S protein of MERS-CoV by RSVR↓SV is mediated by furin during viral egress, whereas the S protein of SARS-CoV is not completely cleaved because SARS-CoV lacks a furin-like cleavage site (SLLR-ST). In MERS-CoV, S protein cleavage occurs at the conserved sequence AYT↓M by the protease (elastase, cathepsin L or TMPRS) expressed by target cells. On the other hand, the S protein of SARS-CoV-2 has 12 additional nucleotides upstream of a single Arg↓ cleavage site 1 forming the PRRAR↓SV sequence, which corresponds to the furin-like cleavage site (SLLR-ST). As described above, the presence of a furin-like cleavage site (SLLR-ST) in SARS-CoV-2 promotes priming of the S protein, and furthermore, enhances SARS-CoV-2 transmissibility compared to SARS-CoV. That is, there is a clear structural difference between SARS-CoV-2 and SARS-CoV.

In addition, SARS-CoV is so lethal that there is no treatment for the disease, while SARS-CoV-2 shows a relatively low lethality rate compared to SARS-CoV, but is highly contagious. Therefore, the need for a preventive vaccine against COVID-19 is very urgent.

Further, the RNA nucleotide sequence of SARS-CoV-2 has a clear difference compared to the RNA nucleotide sequences of the existing SARS-CoV and MERS-CoV. For example, the RNA nucleotide sequence of SARS-CoV-2 differs by 17.7% from the RNA nucleotide sequence of the existing SARS-CoV.
SARS coronavirus (https://www.ncbi.nlm.nih.gov/nuccore/NC_004718.3)
MERS virus (https://www.ncbi.nlm.nih.gov/nuccore/NC_019843.3)
SARS-CoV-2 virus (https://www.ncbi.nlm.nih.gov/nuccore/NC_045512.2)
   It is clear that the difference of 17.7% is such a significant difference that the existing SARS-CoV treatment cannot be used as it is for the prevention and treatment of SARS-CoV-2, and the development of a new vaccine for SARS-CoV-2 is absolutely necessary.

The SARS-CoV-2 has an envelope and consists of a 5′-capping structure and a 3′-poly-A tail, approximately 30 kb long. The viral genome, RNA itself, is a positive single strand RNA that acts as a transcript. The genome of SARS-CoV-2 encodes four structural proteins: spike glycoprotein (S), nucleoprotein (N), membrane protein (M), and envelope protein (E). The S glycoprotein binds specifically to the host cell receptor, the N protein binds to the RNA genome to form a nucleocapsid, the M protein connects between the membrane and the capsid, and the E proteins is involved in the virus assembly and make up the envelope.

The S protein of SARS-CoV-2 is composed of two functional subunits, S1 and S2, and three S proteins form a trimeric fusion structure. The S1 subunit contains a receptor-binding domain (RBD) that binds to angiotensin-converting enzyme 2 (ACE2), the host cell receptor, and the S2 subunit plays a role in allowing viruses to enter the cell through fusion with the cell membrane of the host cell. Therefore, the S protein is used as a main target for the development of a therapeutic agent or vaccine for SARS-CoV-2. However, the comparison of amino acid homology with other coronaviruses shows that the homology in the S1 subunit and RBD of the S protein is relatively low, so caution is needed in the development of a therapeutic agent or vaccine. In addition, SARS-CoV-2 is classified into three groups (A (or S), B (or V), and C (or G)) through genetic analysis, and in the case of group C (or G) found in Europe and the United States, it is reported that the transmissibility is stronger due to mutation of a specific amino acid in the RBD part. Therefore, it is necessary to develop a vaccine or and therapeutic agent that is effective against in genetic mutations. However, there is currently no COVID-19 vaccine that has been developed, and considering the urgency of development, many companies, organizations, and research institutes around the world are developing vaccines using various vaccine platforms. The most advanced vaccines currently under development include inactivated vaccines, virus vectors, and mRNA vaccines. Inactivated vaccines are a system that has been used and verified in traditional vaccine development, but have the disadvantage of having risks and requiring special BSL3 facilities because they directly use pathogens of COVID-19. Viral vectors and mRNA vaccines can be produced quickly, but there are still uncertainties about efficacy and safety as there are no cases of approval as vaccine products. Recombinant protein vaccines have excellent safety, but they show low immunogenicity when administered alone. However, immune efficacy can be improved by using an adjuvant.

The adjuvant is a substance or combination of substances that increase or induce immune responses to vaccine antigens in a desirable direction in order to enhance the clinical effect of vaccines when used together with vaccine antigens. The main function of an adjuvant is to increase and control the immune response to vaccine antigens by directly or indirectly acting on immune stimulation and antigen delivery, or to enhance and improve the clinical efficacy of vaccines, such as extending the duration of defense effects. When infected with pathogenic bacteria or viruses, the surface receptors (pattern recognition receptors) of immune cells recognize the unique pattern (pathogen-associated molecular pattern, PAMP) of pathogenic microorganisms and cause an innate immune response. Toll-like receptors (TLRs) are representative surface receptors, and about 13 types are known in humans. TLR ligands, which respond to tall-like receptors, are being developed as an adjuvant because they directly stimulate immune cells to activate innate immune responses and induce humoral immunity and cell mediated immune responses to vaccine antigens to protect the human body from infectious agents or contribute to tissue recovery.

As a result of analyzing the immunogenicity of COVID-19 patients, it was reported that the humoral immune response derived from the germinal center could not occur due to the absence of the germinal center and reduced T follicular helper cell (Tfh cell) and GCB cell generation (Non-patent reference 2, Naoki Kaneko, et al., 2020, Loss of Bcl-6-expressing T follicular helper cells and germinal centers in COVID-19, cell. 183: 143-157). Therefore, there is a need for a technique that can induce germinal center-derived humoral immune responses. The adjuvant of the present invention is a substance that can improve the humoral immune response by increasing the generation of these T follicular helper cells and germinal center B cells (Non-patent reference 3, Lee B R et al. Combination of TLR1/2 and TLR3 ligands enhances CD4(+) T cell longevity and antibody responses by modulating type I IFN production, SciRep. 2016 Sep. 1, 6:32526), and can function as an effective COVID-19 adjuvant that can overcome the suppression of the humoral immune response by COVID-19. In addition, SARS-CoV-2-specific memory T cells have been reported as an important factor that can prevent viral infection and alleviate the symptoms, so it is necessary to induce not only a humoral immune response but also a cell mediated immune response (Non-patent reference 4, Takuya Sekine, et al., 2020, Robust T cell immunity in convalescent individuals with asymptomatic or mild COVID-19). In addition, the enhancement of the cell mediated immune responses can be a way to solve the symptoms of COVID-19 infection in which the humoral immune response is reduced. The adjuvant of the present invention strongly induces not only a humoral immune response but also a cell mediated immune response, especially a substance that maintains a high frequency of antigen-specific CD4+ T cells in memory phase, and can function as a COVID-19 adjuvant capable of effectively improving vaccines.

Accordingly, the present inventors confirmed that L-pampo, a complex adjuvant of a lipopeptide and poly(I:C), can induce both a humoral immune response and a cell mediated immune response to recombinant COVID-19 antigens through a study on the development of a COVID-19 vaccine. Therefore, the present inventors have completed the present invention by confirming that the vaccine composition containing L-pampo, the adjuvant of the present invention, can be developed as an effective COVID-19 vaccine through improving immunity to recombinant COVID-19 antigens and used commercially.

### SUMMARY OF THE INVENTION

It is an object of the present invention to provide a vaccine composition for preventing or treating coronavirus disease 2019 (COVID-19).

It is another object of the present invention to provide a method for generating an immune response against novel coronavirus in a subject, comprising a step of administering the vaccine composition for preventing or treating coronavirus disease 2019 (COVID-19) to a non-human subject.

It is another object of the present invention to provide a pharmaceutical composition for preventing or treating coronavirus disease 2019 (COVID-19).

To achieve the above objects, in an aspect of the present invention, the present invention provides a vaccine composition for preventing or treating coronavirus disease 2019 (COVID-19), comprising a coronavirus antigen and a vaccine adjuvant including a lipopeptide and poly(I:C).

In another aspect of the present invention, the present invention provides a method for generating an immune response against novel coronavirus in a subject, comprising a step of administering the vaccine composition for preventing or treating coronavirus disease 2019 (COVID-19) to a non-human subject.

In another aspect of the present invention, the present invention provides a pharmaceutical composition for preventing or treating coronavirus disease 2019 (COVID-19), comprising a coronavirus antigen and a vaccine adjuvant including a lipopeptide and poly(I:C).

### Advantageous Effect

The vaccine composition for preventing or treating COVID-19, provided in one aspect of the present invention, can greatly induce both a humoral immune response and a cellular immune response to a recombinant COVID-19 antigen, and thus can be developed as a COVID-19 vaccine so as to be commercially and effectively usable.

### BRIEF DESCRIPTION OF THE DRAWINGS

FIG. 1 is a diagram confirming the COVID-19 S1 antigen-specific antibody titer of the test vaccine prepared with a recombinant corona protein and L-pampo, an adjuvant, in a mouse model.

FIG. 2 is a diagram confirming the COVID-19 RBD antigen-specific antibody titer of the test vaccine prepared with a recombinant corona protein and L-pampo, an adjuvant, in a mouse model.

FIG. 3 is a diagram confirming the COVID-19 NP antigen-specific antibody titer of the test vaccine prepared with a recombinant corona protein and L-pampo, an adjuvant, in a mouse model.

FIG. 4a is a diagram comparing the number of IFN-γ spots generated by the test vaccine prepared with S1 (spike protein 1), a recombinant corona protein, and L-pampo, an adjuvant, in a mouse model using the ELISPOT method for the cell mediated immune response.

FIG. 4b is a diagram comparing the number of IFN-γ spots generated by the test vaccine prepared with RBD (receptor-binding domain), a recombinant corona protein, and L-pampo, an adjuvant, in a mouse model using the ELISPOT method for the cell mediated immune response.

FIG. 4c is a diagram comparing the number of IFN-γ spots generated by the test vaccine prepared with NP (nucleoprotein), a recombinant corona protein, and L-pampo, an adjuvant, in a mouse model using the ELISPOT method for the cell mediated immune response.

FIG. 5a is a diagram comparing the degree of IFN-γ secretion induced by the test vaccine prepared with S1 (spike protein 1), a recombinant corona protein, and L-pampo, an adjuvant, in a mouse model using the cytokine ELISA method for the cell mediated immune response.

FIG. 5b is a diagram comparing the degree of IFN-γ secretion induced by the test vaccine prepared with RBD (receptor-binding domain), a recombinant corona protein, and L-pampo, an adjuvant, in a mouse model using the cytokine ELISA method for the cell mediated immune response.

FIG. 5c is a diagram comparing the degree of IFN-γ secretion induced by the test vaccine prepared with NP (nucleoprotein), a recombinant corona protein, and L-pampo, an adjuvant, in a mouse model using the cytokine ELISA method for the cell mediated immune response.

FIG. 6a is a diagram comparing the induction of COVID-19 S1 antigen-specific antibody titer caused by the test vaccine prepared with a recombinant corona protein and L-pampo or another adjuvant in a mouse model.

FIG. 6b is a diagram comparing the induction of COVID-19 RBD antigen-specific antibody titer caused by the test vaccine prepared with a recombinant corona protein and L-pampo or another adjuvant in a mouse model.

FIG. 7 is a diagram comparing the induction of neutralizing antibodies caused by the test vaccine prepared with a recombinant corona protein and L-pampo or another adjuvant using a method of inhibiting the binding of ACE2 receptors and RBD proteins in a mouse model.

FIG. 8 is a diagram comparing the induction of a cell mediated immune response by the test vaccine prepared with a recombinant corona protein and L-pampo or another adjuvant in a mouse model.

FIG. 9a is a diagram comparing the induction of COVID-19 S1 antigen-specific antibody titers according to the weight ratio of the recombinant corona protein, and the lipopeptide Pam3CSK4 and poly(I:C) in the adjuvant L-pampo in a mouse model.

FIG. 9b is a diagram comparing the induction of COVID-19 RBD antigen-specific antibody titers according to the weight ratio of the recombinant corona protein, and the lipopeptide Pam3CSK4 and poly(I:C) in the adjuvant L-pampo in a mouse model.

FIG. 10 is a diagram comparing the induction of neutralizing antibodies according to the weight ratio of the recombinant corona protein, and the lipopeptide Pam3CSK4 and poly(I:C) in the adjuvant L-pampo in a mouse model.

FIG. 11 is a diagram comparing the induction of cell mediated immune responses according to the weight ratio of the recombinant corona protein, and the lipopeptide Pam3CSK4 and poly(I:C) in the adjuvant L-pampo in a mouse model.

FIG. 12 is a diagram comparing the induction of antibody titers according to the recombinant corona protein and the type of the lipopeptide of the adjuvant L-pampo in a mouse model.

FIG. 13 is a diagram comparing the induction of neutralizing antibodies according to the recombinant corona protein and the type of the lipopeptide of the adjuvant L-pampo in a mouse model.

FIG. 14 is a diagram comparing the induction of cell mediated immune responses according to the recombinant corona protein and the type of the lipopeptide of the adjuvant L-pampo in a mouse model.

### DESCRIPTION OF THE PREFERRED EMBODIMENTS

Hereinafter, the present invention is described in detail.

The embodiments of this invention can be modified in various other forms, and the scope of the present invention is not limited to the embodiments described below. It is well understood by those in the art who has the average knowledge on this field that the embodiments of the present invention are given to explain the present invention more precisely.

In addition, the “inclusion” of an element throughout the specification does not exclude other elements, but may include other elements, unless specifically stated otherwise.

In an aspect of the present invention, the present invention provides a vaccine composition for preventing or treating coronavirus disease 2019 (COVID-19), comprising a coronavirus antigen and a vaccine adjuvant including a lipopeptide and poly(I:C).

Herein, the coronavirus antigen is a novel coronavirus (2019 novel coronavirus, 2019-nCoV) antigen.

More specifically, the antigen may be a viral protein of a spike protein (S) that plays an important role in in vivo infection of SARS-CoV-2, a pathogen of COVID-19, and its components spike protein 1 (S1), receptor-binding domain (RBD) and nucleoprotein (NP), an inactivated virus antigen, or a combination thereof (e.g., a combination of S1 and RBD), but not always limited thereto.

The lipopeptide was first synthesized by J. Metzger et al as a synthetic analogue of a lipopeptide derived from bacteria and mycoplasma (Metzger, J. et al., 1991, Synthesis of novel immunologically active tripalmitoyl-S-glycerylcysteinyllipopeptides as useful intermediates for immunogen preparations. Int. J. Peptide Protein Res. 37: 46-57).

The molecular structure of the compound represented by the following formula 1 is N-palmitoyl-S-[2,3-bis(palmitoyloxy)-(2RS)-propyl]-[R]-cystein-SKKKK(pam3Cys-SKKKK), and various other analogues have been synthesized.

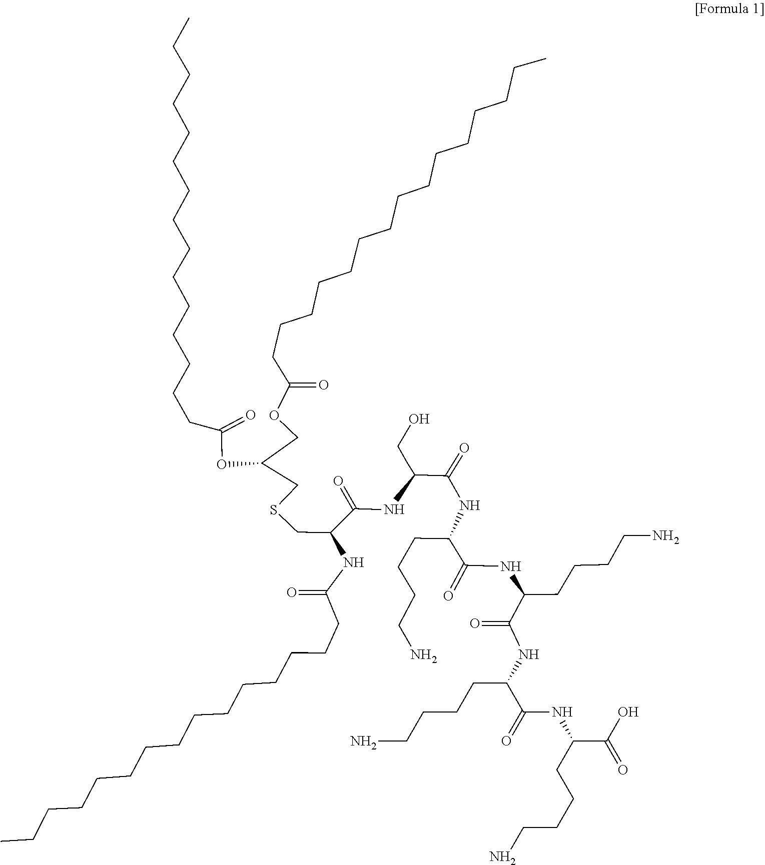 [SMILES: CCCCCCCCCCCCCCCCCC(=O)OC[C@H](CSC[C@H](NC(=O)CCCCCCCCCCCCCCCC)C(=O)N[C@@H](CCO)C(=O)N[C@@H](CCCCN)C(=O)N[C@@H](CCCCN)C(=O)N[C@@H](CCCCN)C(=O)N[C@@H](CCCCN)C(=O)O)OC(=O)CCCCCCCCCCCCCCCC]

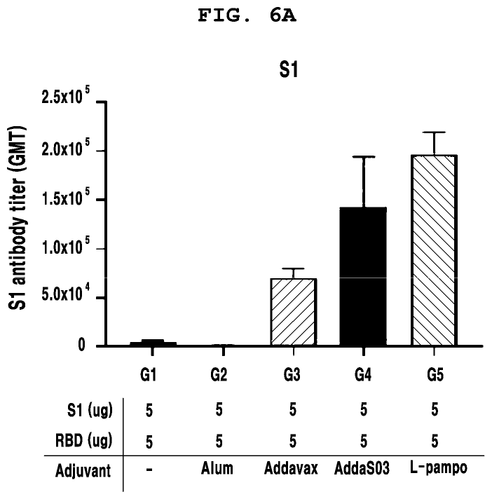

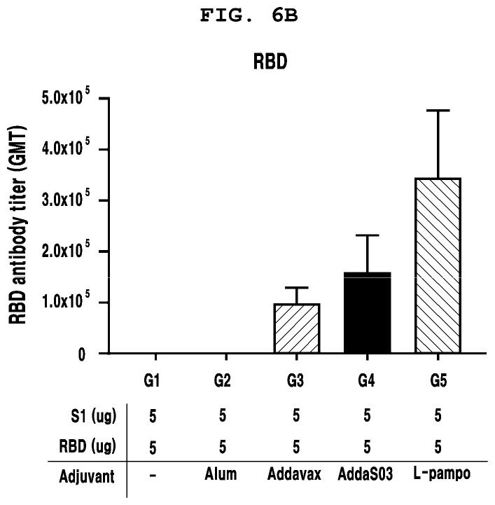

According to H. Schild et al., when Pam3Cys-Ser-Ser was combined with an influenza virus T cell epitope and administered to mice, virus-specific cytotoxic T lymphocytes (CTLs) were induced. In the present invention, it was also confirmed that the SARS-CoV-2 antigen-specific antibody titer of Pam3Cys-SKKKK was relatively superior to that of aluminum hydroxide (see FIGS. 6a and 6b). In general, lipopeptides are known as ligands for TLR2. The use of such lipopeptides is not limited to Pam3Cys-SKKKK, and a lipopeptide can consist of a fatty acid bound to a glycerol molecule and several amino acids. Specific examples thereof include PHC-SKKKK, Ole2PamCys-SKKKK, Pam2Cys-SKKKK, PamCys(Pam)-SKKKK, Ole2Cys-SKKKK, Myr2Cys-SKKKK, PamDhc-SKKKK, PamCSKKKK, Dhc-SKKKK, and the like. The number of fatty acids in a molecule can be one or more. The number of amino acids in a lipopeptide can be one or more. In addition, the fatty acid and amino acid can be chemically modified. Furthermore, the lipopeptide can be a lipoprotein, either as a part of a molecule or as a whole molecule, derived from gram-positive or gram-negative bacteria or mycoplasma.

The poly(I:C) has been used as a potent inducer of type 1 interferon in in vitro and in vivo studies. Moreover, poly(I:C) is known to stably and maturely form dendritic cells, the most potent antigen-presenting cells in mammals (Rous, R. et al 2004. poly(I:C) used for human dendritic cell maturation preserves their ability to secondarily secrete bioactive 11-12, International Immunol. 16: 767-773). According to these previous reports, poly(I:C) is a potent IL-12 inducer, and IL-12 is an important cytokine that induces cellular immune response and formation of IgG2a or IgG2b antibody by driving the immune response to develop Th1. In addition, poly(I:C) is known to have strong adjuvant activity against peptide antigens (Cui, Z. and F. Qui. 2005. Synthetic double stranded RNA poly I:C as a potent peptide vaccine adjuvant: Therapeutic activity against human cervical cancer in a rodent model. Cancer Immunol. Immunotherapy 16: 1-13). Poly (I:C) can have a length in a range of 50 to 5,000 bp, preferably 50 to 2,000 bp, and more preferably 100 to 500 bp, but not always limited thereto.

The lipopeptide and poly(I:C) can be included in the vaccine composition at a weight ratio of 0.1 to 10:1, a weight ratio of 1.25 to 2:1, a weight ratio of 1.25 to 1.5:1, or a weight ratio of 1.25:1, but not always limited thereto. However, the ratio can be adjusted to an appropriate level according to the patient's condition. In addition, the vaccine composition can be an aqueous solution formulation. That is, the vaccine composition can include, as a component, an aqueous solution formulation of a vaccine adjuvant including a coronavirus antigen, a lipopeptide and poly(I:C).

The vaccine composition can further include at least one selected from the group consisting of pharmaceutically acceptable carriers, diluents and adjuvants. For example, the vaccine composition can include a pharmaceutically acceptable carrier, and can be formulated for human or veterinary use and administered through various routes. The vaccine composition may be administered through oral, intraperitoneal, intravenous, intramuscular, subcutaneous, and intradermal routes. Preferably, it is formulated and administered as an injection. Injections can be prepared using aqueous solvents such as physiological saline and Ringer's solution, vegetable oils, higher fatty acid esters (e.g., ethyl oleate, etc.), and non-aqueous solvents such as alcohols (e.g., ethanol, benzyl alcohol, propylene glycol, glycerin, etc.) and can include pharmaceutical carriers such as stabilizers (e.g., ascorbic acid, sodium sulfite, sodium pyrosulfate, BHA, tocopherol, EDTA, etc.) to prevent deterioration, emulsifiers, buffers for pH control, preservatives for preventing microbial development (e.g., chimerosal, benzalkonium chloride, phenol, cresol, benzyl alcohol, etc.) and preservatives (e.g., phenylmercuric nitrate, thimerosal, benzalkonium chloride, phenol, cresol, benzyl alcohol, etc.) to inhibit microbial growth. The vaccine composition can be administered in a pharmaceutically effective amount. At this time, the term “pharmaceutically effective amount” means an amount sufficient to exhibit a vaccine effect but an amount not to cause side effects or serious or excessive immune responses. The exact dosage concentration depends on the antigen to be administered, and can be easily determined by those skilled in the art according to factors well known in the medical field, such as the patient's age, weight, health, gender, sensitivity to drugs, administration route, and administration method. The composition of the present invention can be administered once or several times.

The vaccine composition exhibits excellent protective immunity against novel coronavirus. In another aspect of the present invention, the present invention provides a method for generating an immune response against novel coronavirus in a subject, comprising a step of administering the vaccine composition for preventing or treating coronavirus disease 2019 (COVID-19) to a non-human subject.

If the vaccine composition is administered to a human (patient), it can be administered in an amount effective to stimulate an immune response in vivo, for example, it can be administered to humans once or several times, and the dosage is 1-250 μg, more preferably 10-100 μg, but not always limited thereto.

In another aspect of the present invention, the present invention provides a pharmaceutical composition for preventing or treating coronavirus disease 2019 (COVID-19), comprising a coronavirus antigen and a vaccine adjuvant including a lipopeptide and poly(I:C).

In another aspect of the present invention, the present invention provides a method for preventing, ameliorating or treating coronavirus disease 2019 (COVID-19), comprising a step of administering a pharmaceutical composition containing a coronavirus antigen and a vaccine adjuvant including a lipopeptide and poly(I:C) to a subject.

In another aspect of the present invention, the present invention provides a use of a pharmaceutical composition comprising a coronavirus antigen and a vaccine adjuvant including a lipopeptide and poly(I:C) for preventing, ameliorating or treating coronavirus disease 2019 (COVID-19).

In another aspect of the present invention, the present invention provides a use of a pharmaceutical composition comprising a coronavirus antigen and a vaccine adjuvant including a lipopeptide and poly(I:C) for the preparation of a medicament for preventing, ameliorating or treating coronavirus disease 2019 (COVID-19).

The vaccine composition for preventing or treating coronavirus disease 2019 (COVID-19), provided in one aspect of the present invention, can greatly induce both a humoral immune response and a cellular immune response to a recombinant COVID-19 antigen, and thus can be developed as a COVID-19 vaccine so as to be commercially and effectively usable.

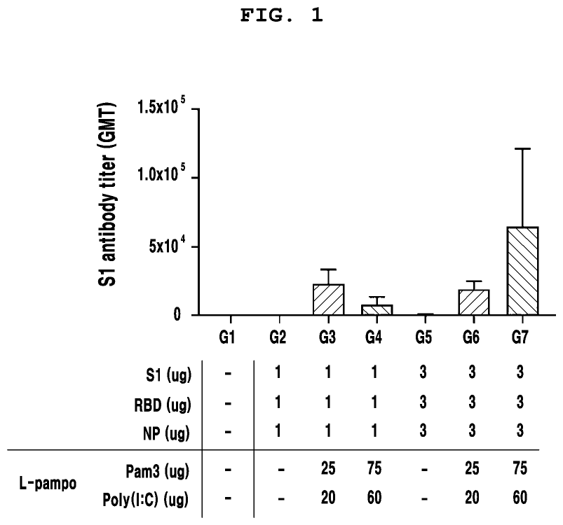

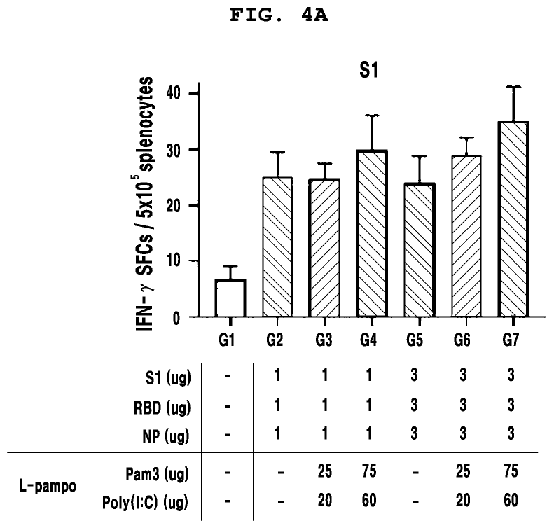

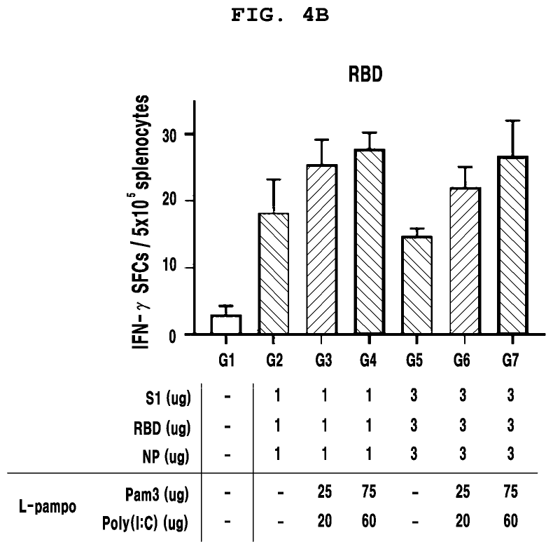

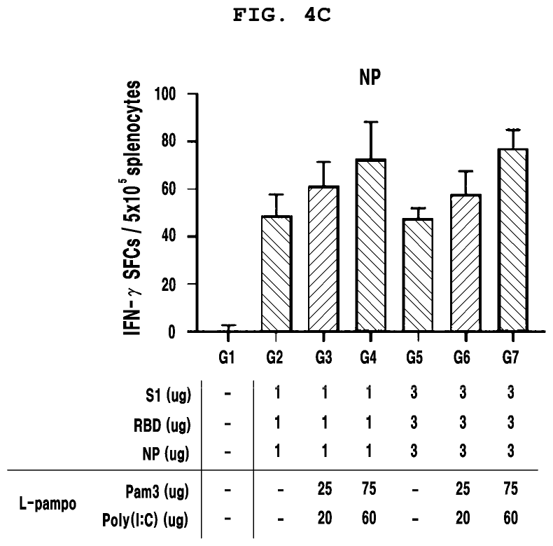

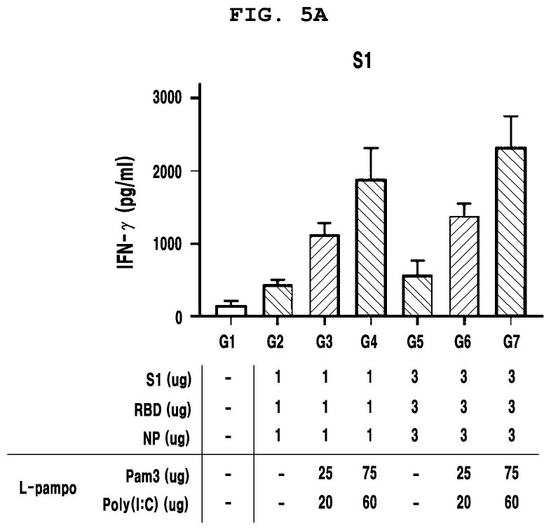

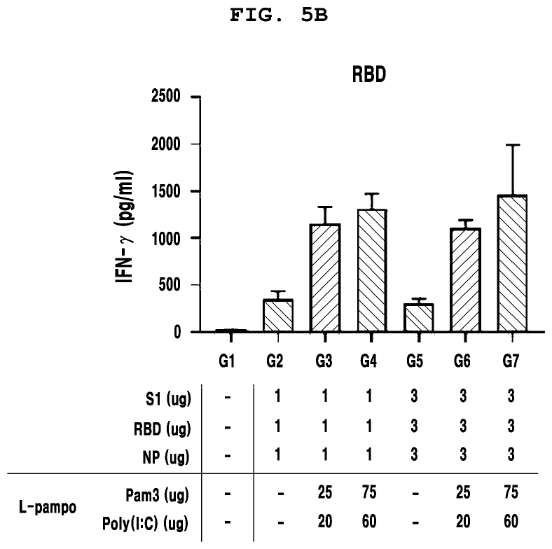

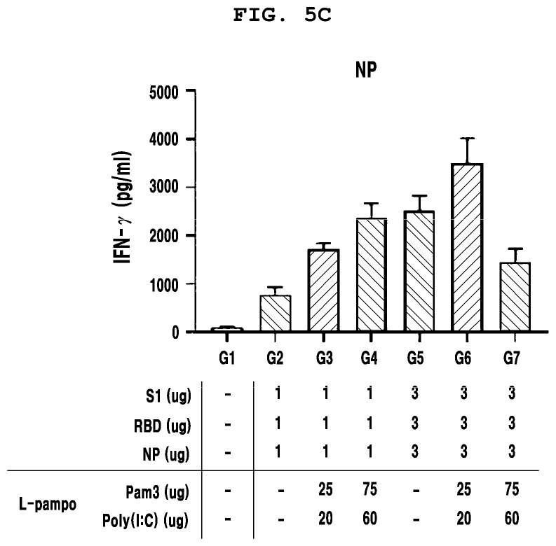

Specifically, antigen-specific antibody titers were investigated in a mouse model using the test vaccine prepared to include S1, RBD, and NP proteins, which are antigens of COVID-19, and Pam3CSK4, a lipopeptide, and poly(I:C). As a result, it was confirmed that the vaccine composition provided in one aspect of the present invention showed significantly higher antibody titers specific to S1, RBD, and NP proteins compared to the test group using an antigen alone (see FIGS. 1 to 3). In addition, the cellular immune response to the test vaccine prepared above was analyzed. As a result, it was confirmed that the vaccine composition provided in an aspect of the present invention induced more IFN-γ secreting cells specific to the COVID-19 antigens S1, RBD, and NP compared to the test group using an antigen alone (see FIGS. 4a to 4c and 5a to 5c). From the above results, it was confirmed that the vaccine composition provided in one aspect of the present invention can be used as an effective COVID-19 vaccine by generating high antigen-specific antibodies and inducing a strong antigen-specific cellular immune response.

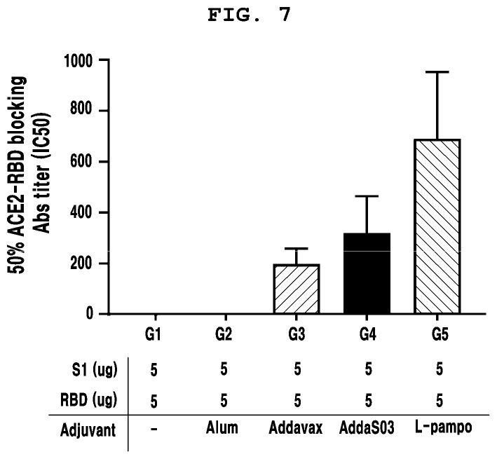

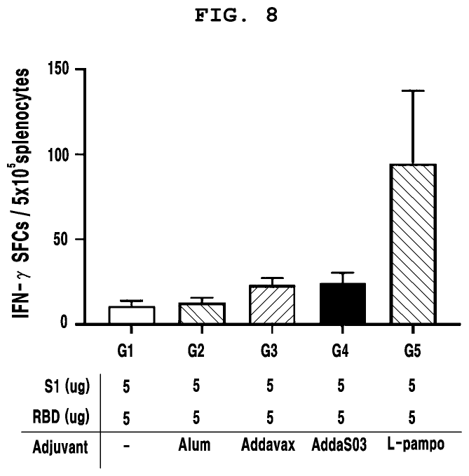

In addition, antigen-specific antibody titers were investigated in a mouse model using the test vaccine prepared to include S1, RBD, and NP proteins, which are antigens of COVID-19, and Pam3CSK4, a lipopeptide, and poly(I:C) in comparison with a control group in which only the type of adjuvant was different. As a result, it was confirmed that the vaccine composition provided in one aspect of the present invention showed significantly higher antibody titers specific to S1 and RBD proteins compared to the test vaccine using Alum and oil emulsion as an adjuvant (see FIGS. 6a and 6b). In addition, the neutralizing antibody efficacy was investigated for the test vaccine and the control test vaccine. As a result, it was confirmed that the vaccine composition provided in one aspect of the present invention induced neutralizing antibodies higher than the control group in which Alum and oil emulsion were mixed (see FIG. 7). In addition, the cellular immune response was evaluated for the test vaccine and the control test vaccine. As a result, it was confirmed that the most IFN-γ spot formation was induced by the test vaccine including Pam3CSK4 and poly(I:C) as an adjuvant compared to the control group in which Alum and oil emulsion were mixed (see FIG. 8). From the above results, it was confirmed that the vaccine composition provided in one aspect of the present invention is a COVID-19 vaccine composition that may exhibit strong immune efficacy compared to other vaccine compositions including Alum and oil emulsion as an adjuvant.

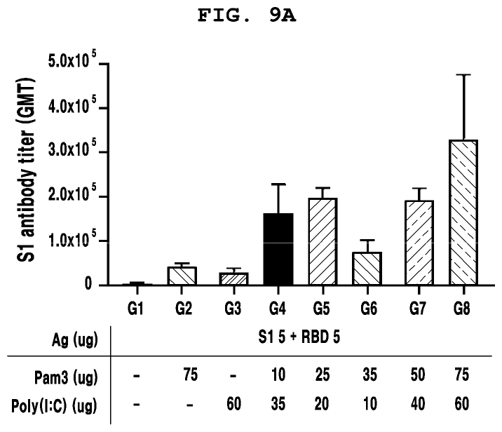

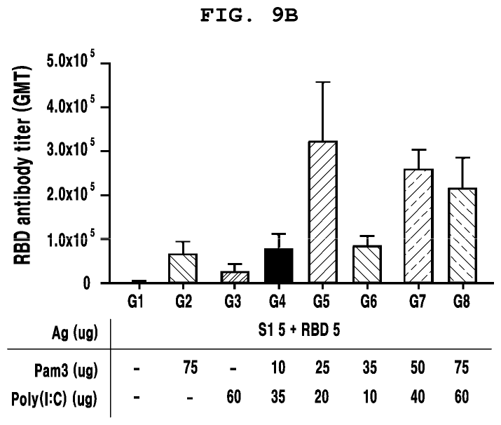

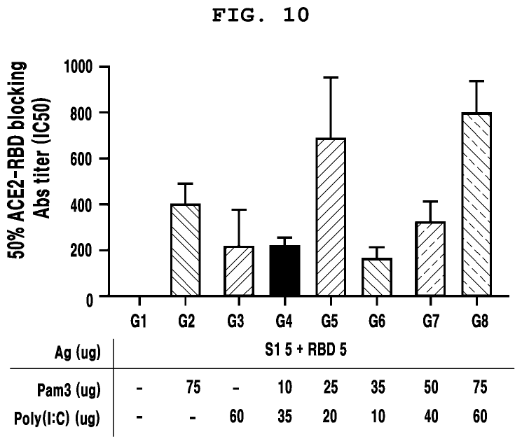

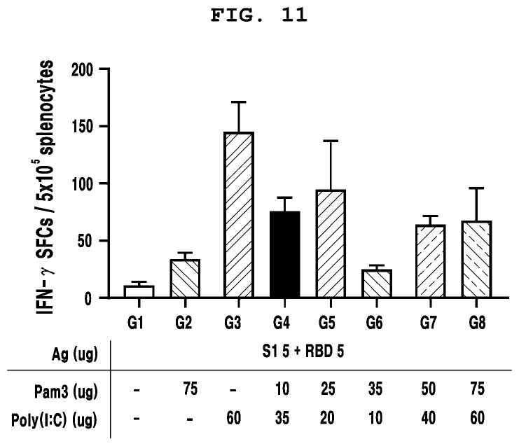

In addition, antigen-specific antibody titers were investigated in a mouse model using the test vaccine prepared to include S1, RBD, and NP proteins, which are antigens of COVID-19, and Pam3CSK4, a lipopeptide, and poly(I:C) in which the weight ratios of the lipopeptide Pam3CSK4 and poly(I:C) are varied. As a result, the test vaccine containing Pam3CSK4 and poly(I:C) as an adjuvant induced an antibody titer specific to S1 and RBD proteins higher than the test vaccine using either Pam3CSK4 or poly(I:C) alone, and the test vaccine using Pam3CSK4 and poly(I:C) at a weight ratio of 1.25:1 showed the highest antibody titer (see FIGS. 9a and 9b). In addition, as a result of confirming the induction of neutralizing antibodies, it was confirmed that the highest level of neutralizing antibodies was induced by the test vaccine using Pam3CSK4 and poly(I:C) at a weight ratio of 1.25:1 (FIG. 10). In addition, as a result of evaluating the cellular immune response, the most IFN-γ spot formation was induced by the test vaccine using only poly(I:C), and the test vaccine using Pam3CSK4 and poly(I:C) at a weight ratio of 1.25:1 induced more IFN-γ spot formation than the test vaccines using other weight ratios (FIG. 11). From the above results, it was confirmed that the vaccine composition provided in one aspect of the present invention exhibits potent humoral immune efficacy compared to the vaccine composition comprising only one of the lipopeptide Pam3CSK4 or poly(I:C), and in particular, the test vaccine comprising Pam3CSK4 and poly(I:C) at a weight ratio of 1.25:1 is a COVID-19 vaccine composition that induces potent humoral and cellular immune responses.

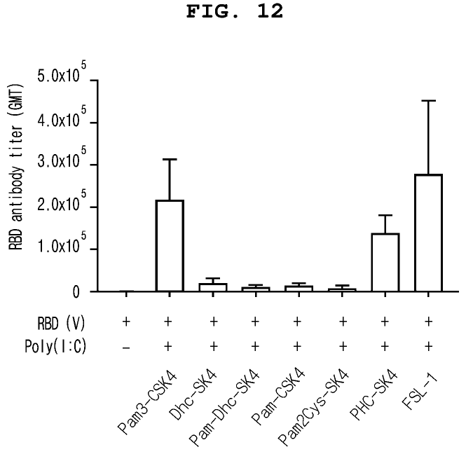

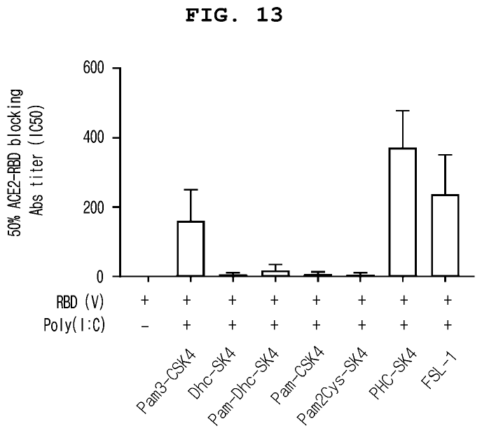

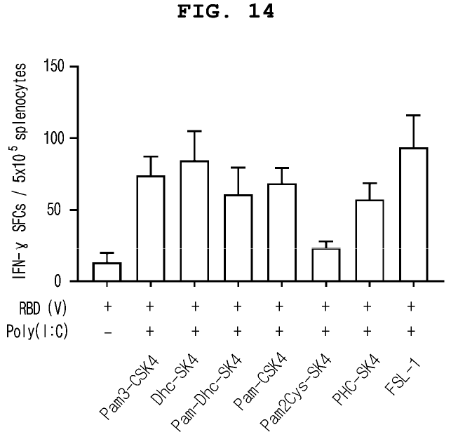

In addition, antigen-specific antibody titers were investigated in a mouse model using the test vaccine prepared to include RBD, a South African mutant antigen of COVID-19, different types of lipopeptide, and poly(I:C). As a result, the formulation containing Pam3CSK4 or FSL-1 lipopeptide and poly(I:C) showed the highest antibody titer, and the formulation containing PHC-SK4 lipopeptide and poly(I:C) also induced high antibody titer (FIG. 12). In addition, as a result of confirming the induction of neutralizing antibodies, It was confirmed that the formulation containing PHC-SK4 lipopeptide and poly(I:C) showed the highest neutralizing antibody titer, and the formulation containing FSL-1 or Pam3CSK4 lipopeptide and poly(I:C) also induced high neutralizing antibodies (FIG. 13). In addition, as a result of evaluating the cellular immune response, it was confirmed that the formation of many IFN-γ spots was induced by all formulations including poly(I:C) and other lipopeptides except for Pam2Cys-SK4 lipopeptide (FIG. 14). From the above results, it was confirmed that the vaccine composition provided in one aspect of the present invention shows high cellular immunity efficacy, and in particular, that the vaccine composition containing Pam3CSK4, PHC-SK4 or FSL-1 lipopeptide and poly(I:C) is a COVID-19 vaccine composition that induces strong humoral and cellular immune responses.

Hereinafter, the present invention will be described in detail by the following examples.

However, the following examples are only for illustrating the present invention, and the contents of the present invention are not limited thereto.

### Example 1: Confirmation of Efficacy of COVID-19 Vaccine in Mouse Model

### <1-1> Preparation and Administration of Test Vaccine

The test vaccine was prepared by mixing 1 μg (or 3 μg) each of S1 (spike protein 1), a COVID-19 antigen, RBD (receptor-binding domain) and NP (nucleoprotein), and then including L-pampo consisting of 25 μg (or 75 jig) of Pam3CSK4 and 20 μg (or 60 μg) of poly(I:C) in the mixture. Each test vaccine was intramuscularly injected twice at 2-week intervals into 6-week-old female Balb/c mice (Orient Bio, Korea) using 1 μg or 3 μg of each antigen per dose.

<1-2> Antigen-Specific Antibody Titer Analysis

In order to analyze the antigen-specific antibody induction efficacy of the test vaccine prepared and administered by the method of Example <1-1>, serum was separated at 2 weeks after completion of immunization, and antigen-specific antibody formation was measured by ELISA (enzyme-linked immunosorbent assay) to determine antibody titer.

Specifically, to analyze antibodies, a 96-well microplate was coated with S1, RBD and NP antigens at a concentration of 0.1 μg/well, respectively, and then reacted with 1% bovine serum albumin for 1 hour to prevent nonspecific binding. After washing the microplate, serially diluted serum was added to each well and reacted at 37° C. for 2 hours. As a secondary antibody, anti-mouse goat IgG-HRP (goat anti-mouse IgG, Sigma, USA) was added thereto and reacted at room temperature for 1 hour. The reacted microplate was washed, a coloring reagent TMB (3,3′,5,5′-Tetramethylbenzidine) peroxidase substrate (KPL, USA) was added, reacted at room temperature, and then O.D (optical density) values were measured at 450 nm using an ELISA reader. The antibody titer was defined as the reciprocal of the antibody dilution multiple representing the O.D value compared to the negative control group.

FIG. 1 shows the results of confirming the COVID-19 S1 antigen-specific antibody titer of the test vaccine prepared with a recombinant corona protein and L-pampo, an adjuvant, in a mouse model.

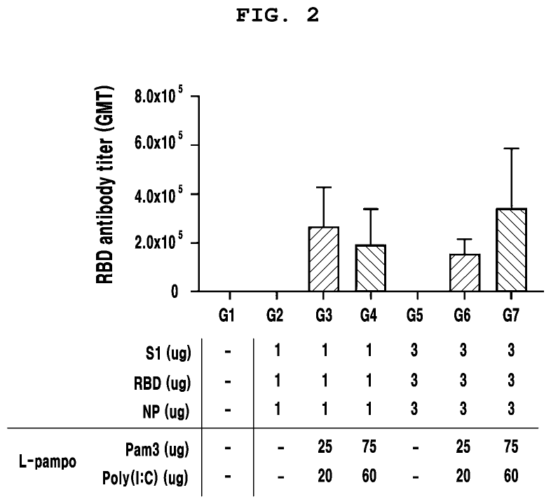

FIG. 2 shows the results of confirming the COVID-19 RBD antigen-specific antibody titer of the test vaccine prepared with a recombinant corona protein and L-pampo, an adjuvant, in a mouse model.

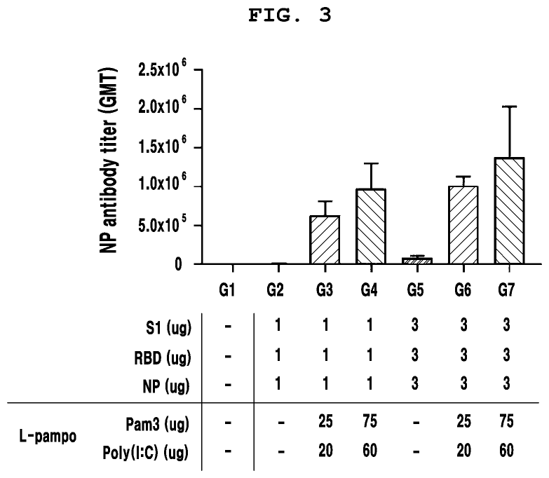

FIG. 3 shows the results of confirming the COVID-19 NP antigen-specific antibody titer of the test vaccine prepared with a recombinant corona protein and L-pampo, an adjuvant, in a mouse model.

As shown in FIG. 1, the S1-specific antibody titer was higher in the L-pampo test groups using the antigen together with Pam3CSK4 and poly(I:C) than in the test groups G2 and G5 using the antigen alone. In addition, the highest antibody titer was shown in the group G7 administered with the vaccine prepared by mixing 3 μg of each of the antigens S1, RBD and NP with 75 μg of Pam3CSK4 and 60 μg of poly(I:C).

As shown in FIGS. 2 and 3, the RBD or NP-specific titer showed similar tendencies as in FIG. 1, and the titer was the highest in the group G7 administered with the vaccine prepared by mixing 3 μg of each of the antigens S1, RBD and NP with 75 μg of Pam3CSK4 and 60 μg of poly(I:C).

### <1-3> Cellular Immune Response Analysis

In order to analyze the cellular immune response caused by the test vaccine prepared and administered by the method of Example <1-1>, the spleen was extracted at 2 weeks after completion of immunization, and all splenocytes were isolated, followed by performing ELISPOT (enzyme linked immuno-spot) and cytokine ELISA.

Specifically, for ELISPOT, an ELISPOT plate to which an antibody against IFN-γ was attached was first washed with PBS, and then complete medium was added thereto to activate the plate. Mouse splenocytes were seeded on the ELISPOT plate at the density of 5×10 5 cells/well, and each COVID-19 antigen (S1, RBD or NP) was added thereto, followed by reaction in a 37° C., 5% CO₂ incubator for 24 hours. Then, the splenocytes were removed, the plate was washed with PBS, and the biotin-conjugated antibody in the Mouse IFN-γELISpot^{PLUS} kit (Mabtech, Sweden) was diluted with PBS containing 0.5% FBS and added to each well of the plate. The plate was reacted at room temperature for 2 hours, washed, and horseradish peroxidase (HRP)-conjugated streptavidin was added to each well of the plate, followed by reaction at room temperature for 1 hour. After washing the plate, a TMB (3,3′,5,5′-tetramethylbenzidine) color reagent was added and reacted until a clear spot appeared, and when the reaction was completed, tertiary distilled water was added to terminate the reaction. The plate was washed several times with distilled water, dried at room temperature, and spots were counted using an ELISPOT reader.

On the other hand, for the sample for performing cytokine ELISA, mouse splenocytes were seeded on a 96 well plate at the density of 1.5×10⁶ cells/well, and each COVID-19 antigen (S1, RBD or NP) was added thereto, followed by reaction in a 37° C., 5% CO₂ incubator for 48 hours. The culture solution of each subject was transferred to a tube, and the supernatant was obtained by centrifugation at 4° C., 3000 rpm for 5 minutes. The antibody for coating included in the Mouse IFN(interferon)-γ ELISA kit (BD, USA) was diluted in a coating buffer, dispensed into a 96-well plate, and the plate was coated at 37° C. for 2 hours. After washing the plate, 10% FBS (fetal bovine serum) was added thereto, followed by blocking at 37° C. for 1 hour. After washing the plate, the standard solution and the sample (splenocyte culture medium) obtained above were dispensed into each well and reacted at room temperature for 2 hours. After washing the plate, a working detector containing a mixture of biotin-conjugated antibody and HRP-conjugated streptavidin was dispensed into the plate, followed by reaction at room temperature for 1 hour. After washing the plate, a TMB color reagent was added and reacted at room temperature for 5 to 10 minutes. Then, the color development reaction was terminated using a stop solution, and the O.D. values were measured at 450 nm using an ELISA reader. A mathematical formula was created by drawing a calibration curve using the values of the standard solution, and the amount of IFN-γ secretion in the test sample was calculated based on this.

FIGS. 4a to 4c show the results of confirming the cellular immune response caused by the test vaccine prepared with a recombinant corona protein (S1, RBD or NP) and L-pampo, an adjuvant, in a mouse model by the IFN-γ spot analysis method using the ELISPOT method.

FIGS. 5a to 5c show the results of confirming the cellular immune response caused by the test vaccine prepared with a recombinant corona protein (S1, RBD or NP) and L-pampo, an adjuvant, in a mouse model by the IFN-γ secretion analysis method using the ELISA method.

As shown in FIGS. 4a to 4c, as a result of the IFN-γ ELISPOT analysis, IFN-γ secreting cells specific to COVID-19 antigens (S1, RBD and NP) were more induced in the L-pampo test groups using the antigen together with Pam3CSK4 and poly(I:C) than in the test group using the antigen alone.

In addition, as shown in FIGS. 5a to 5c, as a result of the IFN-γ ELISA analysis, IFN-γ secretion specific to COVID-19 antigens (S1, RBD and NP) was strongly induced in the L-pampo test groups using the antigen together with Pam3CSK4 and poly(I:C) compared to the test group using the antigen alone. In particular, when a vaccine prepared by mixing 75 μg of Pam3CSK4 and 60 μg of poly(I:C) was administered, a significantly high cellular immune response was induced regardless of the antigen concentration.

Therefore, from the results of [Example 1], it was confirmed that the COVID-19 vaccine composition containing the adjuvant of the present invention can be used as an effective COVID-19 vaccine by producing high antigen-specific antibodies and strongly inducing antigen-specific cellular immune responses.

### Example 2: Comparison of Effectiveness of Adjuvant L-Pampo with Other Adjuvants

### <2-1> Preparation and Administration of Test Vaccine

The test vaccine was prepared by mixing 5 μg each of S1 and RBD proteins, which are antigens of COVID-19, and then including L-pampo, the adjuvant of the present invention or other adjuvants (Alum, Addavax and AddaS03) in the mixture. Addavax (MF59-like adjuvant) and AddaS03 (A503-like adjuvant) were used to compare the efficacy of the currently commercialized oil emulsion adjuvant and the adjuvant of the present invention.

More specifically, the test vaccine containing the adjuvant of the present invention was prepared to contain 5 μg of S1 and RBD antigens, and L-pampo [75 μg of Pam3CSK4 and 60 μg of poly(I:C)], respectively. The test vaccine containing the Alum adjuvant was prepared to contain 5 μg of S1 and RBD antigens and 100 μg of Alum, respectively. The test vaccine containing the Addavax adjuvant was prepared by mixing each of 5 μg of S1 and RBD antigens and Addavax (1:1, v/v).

Each test vaccine was intramuscularly injected twice at 3-week intervals into Balb/c mice using 5 μg of each antigen per dose.

### <2-2> Antigen-Specific Antibody Titer Analysis

In order to analyze the antigen-specific antibody induction efficacy of the test vaccine prepared and administered by the method of Example <2-1>, serum was separated at 2 weeks after the second immunization, and antigen-specific antibody formation was measured by ELISA to determine antibody titer.

FIGS. 6a and 6b show the results of comparing the antibody titer induction caused by the test vaccine prepared with the recombinant corona protein S1, RBD and the adjuvant L-pampo and the test vaccine prepared with the oil emulsion adjuvant in a mouse model.

As shown in FIGS. 6a and 6b, it was found that the antigen-specific antibody titer was increased in all experimental groups. The highest antibody titer was found to be induced by the experimental group containing L-pampo [75 μg of Pam3CSK4 and 60 μg of poly(I:C)] compared to the experimental group containing the oil emulsion adjuvant. Therefore, it was confirmed that the adjuvant L-pampo of the present invention significantly improves the formation of antibodies against the SARS-CoV-2 antigen compared to the oil emulsion adjuvant.

### <2-3> Neutralizing Antibody Induction Analysis

In order to analyze the induction of neutralizing antibodies by the test vaccine prepared and administered by the method of Example <2-1>, mouse serum was separated at 2 weeks after the second immunization, and analyzed using a method of inhibiting binding of ACE2 (angiotensin-converting enzyme 2) receptor and RBD protein.

Specifically, to perform the method of inhibiting binding of ACE2 receptor and RBD protein, a 96-well microplate was coated with RBD antigen at a concentration of 0.1 μg/well and then reacted with 1% bovine serum albumin for 2 hours to prevent non-specific binding. After washing the microplate, serially diluted serum was added to each well and reacted at room temperature for 2 hours. HRP-conjugate human ACE2 protein was added thereto and reacted at room temperature for 1 hour. The reacted microplate was washed, a coloring reagent TMB (3,3′,5,5′-Tetramethylbenzidine) peroxidase substrate (KPL, USA) was added, reacted at room temperature, and then O.D values were measured at 450 nm using an ELISA reader. The antibody titer inhibiting the binding of ACE2 receptor and RBD protein by 50% was defined as the reciprocal of the antibody dilution multiple representing 50% of the O.D value compared to the negative control group.

FIG. 7 shows the results of comparing the induction of neutralizing antibodies caused by the test vaccine prepared with a recombinant corona protein and the L-pampo adjuvant and the test vaccine prepared with another adjuvant in a mouse model.

As shown in FIG. 7, the highest antibody titer inhibiting the binding of ACE2 receptor and RBD protein was induced by the experimental group containing L-pampo [75 μg of Pam3CSK4 and 60 μg of poly(I:C)] compared to the experimental group containing the oil emulsion adjuvant. Therefore, it was confirmed that the adjuvant of the present invention significantly improves the formation of neutralizing antibodies against SARS-CoV-2 compared to the oil emulsion adjuvant.

### <2-4> Cellular Immune Response Analysis

In order to analyze the cellular immune response caused by the test vaccine prepared and administered by the method of Example <2-1>, the spleen was extracted at 2 weeks after the second immunization, and all splenocytes were isolated, followed by performing ELISPOT.

FIG. 8 shows the results of comparing the induction of a cell mediated immune response by the test vaccine prepared with a recombinant corona protein and the L-pampo adjuvant and the test vaccine prepared with the oil emulsion adjuvant in a mouse model.

As shown in FIG. 8, the most IFN-γ spot formation was induced by the experimental group containing L-pampo [75 μg of Pam3CSK4 and 60 μg of poly(I:C)] compared to the experimental group containing the oil emulsion adjuvant.

Therefore, from the results of [Example 2], it was confirmed that the vaccine composition including the L-pampo adjuvant of the present invention is a COVID-19 vaccine composition capable of exhibiting strong immune efficacy compared to the vaccine composition containing the Alum or oil emulsion adjuvant.

### Example 3: Confirmation of Vaccine Efficacy According to Weight Ratio of Lipopeptide and Poly(I:C) in L-Pampo

### <3-1> Preparation and Administration of Test Vaccine

The test vaccine was prepared by mixing 5 μg each of S1 and RBD proteins, which are antigens of COVID-19, and then including the adjuvant L-pampo of the present invention prepared in various weight ratios of the lipopeptide Pam3CSK4 to poly(I:C), such as 1.25 to 3.5:1 or 1:3.5. In addition, the test vaccine containing 75 μg of high dose Pam3CSK4 or 60 μg of poly(I:C) was prepared, respectively.

Each test vaccine was intramuscularly injected twice at 3-week intervals into Balb/c mice.

### <3-2> Antigen-Specific Antibody Titer Analysis

In order to analyze the antigen-specific antibody induction efficacy of the test vaccine prepared and administered by the method of Example <3-1>, serum was separated at 2 weeks after the second immunization, and antigen-specific antibody formation was measured by ELISA to determine antibody titer.

FIGS. 9a and 9b show the results of comparing the induction of antibody titers according to the weight ratio of the recombinant corona protein, and the lipopeptide Pam3CSK4 and poly(I:C) in the adjuvant L-pampo in a mouse model.

As shown in FIGS. 9a and 9b, it was found that the antigen-specific antibody titer was increased in all experimental groups. The experimental group containing L-pampo was found to induce higher antibody titers than the experimental group containing 75 μg of Pam3CSK4 or 60 μg of poly(I:C), respectively. In addition, it was confirmed that the experimental group containing the L-pampo prepared with Pam3CSK4 and poly(I:C) at a weight ratio of 1.25:1 induced the highest antibody titer compared to the experimental group containing the L-pampo prepared at a different weight ratio.

### <3-3> Neutralizing Antibody Induction Analysis

In order to analyze the induction of neutralizing antibodies by the test vaccine prepared and administered by the method of Example <3-1>, mouse serum was separated at 2 weeks after the second immunization, and analyzed using a method of inhibiting binding of ACE2 receptor and RBD protein.

FIG. 10 shows the results of comparing the induction of neutralizing antibodies according to the weight ratio of the recombinant corona protein, and the lipopeptide Pam3CSK4 and poly(I:C) used as an adjuvant in a mouse model.

As shown in FIG. 10, the highest ACE2 receptor and RBD protein binding inhibitory antibody titer was shown in the experimental group containing the L-pampo prepared with Pam3CSK4 and poly(I:C) at a weight ratio of 1.25:1, confirming high neutralizing antibody induction.

### <3-4> Cellular Immune Response Analysis

In order to analyze the cellular immune response caused by the test vaccine prepared and administered by the method of Example <3-1>, the spleen was extracted at 2 weeks after the second immunization, and all splenocytes were isolated, followed by performing ELISPOT.

FIG. 11 shows the results of comparing the induction of cell mediated immune responses according to the weight ratio of the recombinant corona protein, and the lipopeptide Pam3CSK4 and poly(I:C) used as an adjuvant in a mouse model.

As shown in FIG. 11, the most IFN-γ spot formation was induced by the experimental group containing the L-pampo prepared with Pam3CSK4 and poly(I:C) at a weight ratio of 1.25:1 compared to the experimental group containing the L-pampo prepared at a different weight ratio.

Therefore, from the results of [Example 3], it was confirmed that the vaccine composition containing the adjuvant L-pampo of the present invention including the lipopeptide Pam3CSK4 and poly(I:C) showed higher immune efficacy than the vaccine composition containing either the lipopeptide Pam3CSK4 or poly(I:C). In particular, it was confirmed that the vaccine composition containing the L-pampo prepared with Pam3CSK4 and poly(I:C) at a weight ratio of 1.25:1 is a COVID-19 vaccine composition showing the strongest immune efficacy.

### Example 4: Confirmation of COVID-19 Vaccine Efficacy According to Lipopeptide Type

### <4-1> Preparation and Administration of Test Vaccine

The test vaccine was prepared to include 3.65 μg of RBD, a South African mutant antigen of COVID-19, 20 μg of poly(I:C) and 25 μg each of various lipopeptides such as Pam3CSK4, Dhc-SK4, Pam-Dhc-SK4, Pam2Cys-SK4, PHC-SK4 and FSL-1, which are components of the adjuvant L-pampo of the present invention.

Each test vaccine was intramuscularly injected twice at 3-week intervals into Balb/c mice.

### <4-2> Antigen-Specific Antibody Titer Analysis

In order to analyze the antigen-specific antibody induction efficacy of the test vaccine prepared and administered by the method of Example <4-1>, serum was separated at 2 weeks after the second immunization, and antigen-specific antibody formation was measured by ELISA to determine antibody titer.

FIG. 12 shows the results of comparing the induction of antibody titers according to the recombinant corona protein and the type of the lipopeptide of the adjuvant L-pampo in a mouse model.

As shown in FIG. 12, all of the experimental groups containing the adjuvant L-pampo including various lipopeptides and poly(I:C) showed higher antibody titers than the experimental group containing the antigen alone. Among them, it was confirmed that the experimental group containing the L-pampo prepared with Pam3CSK4, PHC-SK4 or FSL-1 induced a higher antibody titer than the experimental group containing the L-pampo prepared with other lipopeptides.

### <4-3> Neutralizing Antibody Induction Analysis

In order to analyze the induction of neutralizing antibodies by the test vaccine prepared and administered by the method of Example <4-1>, mouse serum was separated at 2 weeks after the second immunization, and analyzed using a method of inhibiting binding of ACE2 receptor and RBD protein.

FIG. 13 shows the results of comparing the induction of neutralizing antibodies according to the recombinant corona protein and the type of the lipopeptide of the adjuvant L-pampo in a mouse model.

As shown in FIG. 13, all of the experimental groups containing the adjuvant L-pampo including various lipopeptides and poly(I:C) showed higher ACE2 receptor and RBD protein binding inhibitory antibody titer than the experimental group containing the antigen alone. Among them, it was confirmed that the experimental group containing the L-pampo prepared with Pam3CSK4, PHC-SK4 or FSL-1 induced higher ACE2 receptor and RBD protein binding inhibitory antibody titer than the experimental group containing the L-pampo prepared with other lipopeptides, confirming high neutralizing antibody induction.

### <4-4> Cellular Immune Response Analysis

In order to analyze the cellular immune response caused by the test vaccine prepared and administered by the method of Example <4-1>, the spleen was extracted at 2 weeks after the second immunization, and all splenocytes were isolated, followed by performing ELISPOT.

FIG. 14 shows the results of comparing the induction of cell mediated immune responses according to the recombinant corona protein and the type of the lipopeptide of the adjuvant L-pampo in a mouse model.

As shown in FIG. 14, all of the experimental groups containing the adjuvant L-pampo including various lipopeptides and poly(I:C) induced more IFN-γ spot formation than the experimental group containing the antigen alone. In addition, the experimental group containing the L-pampo prepared with other lipopeptides except Pam2Cys-SK4 induced the formation of many IFN-γ spots.

Therefore, from the results of [Example 4], it was confirmed that the vaccine composition containing the adjuvant L-pampo of the present invention including various lipopeptides and poly(I:C) showed high immune efficacy. In particular, it was confirmed that the vaccine composition containing the L-pampo prepared with Pam3CSK4, PHC-SK4 or FSL-1 is a COVID-19 vaccine composition showing strong immune efficacy.

1. A vaccine composition for preventing or treating coronavirus disease 2019 (COVID-19), comprising:
a coronavirus antigen and a vaccine adjuvant including a lipopeptide and poly(I:C),
wherein the coronavirus antigen is one or more antigens selected from the group consisting of spike protein 1 (S1), receptor-binding domain (RBD) and nucleoprotein (NP) of SARS-CoV-2 virus,
wherein the lipopeptide is at least one selected from the group consisting of Pam3Cys-SKKKK, PHC-SKKKK, Ole2PamCys-SKKKK, Pam2Cys-SKKKK, PamCys(Pam)-SKKKK, Ole2Cys-SKKKK, Myr2Cys-SKKKK, PamDhc-SKKKK, PamCSKKKK and Dhc-SKKKK, and
wherein the lipopeptide and poly(I:C) are in a weight ratio of 1 to 2:1.

2.-4. (canceled)

5. The vaccine composition for preventing or treating coronavirus disease 2019 (COVID-19) according to claim 1, wherein the vaccine composition is an aqueous solution formulation.

6. The vaccine composition for preventing or treating coronavirus disease 2019 (COVID-19) according to claim 1, wherein the vaccine composition further includes at least one selected from the group consisting of pharmaceutically acceptable carriers, diluents and adjuvants.

7. The vaccine composition for preventing or treating coronavirus disease 2019 (COVID-19) according to claim 1, wherein the vaccine composition is administered through any one administration route selected from the group consisting of oral, transdermal, intramuscular, intraperitoneal, intravenous, subcutaneous and nasal administration.

8. The vaccine composition for preventing or treating coronavirus disease 2019 (COVID-19) according to claim 1, wherein the vaccine composition exhibits protective immunity against novel coronavirus.

9. A method for generating an immune response against novel coronavirus in a subject, comprising a step of administering the vaccine composition for preventing or treating coronavirus disease 2019 (COVID-19) of claim 1 to a non human subject.

10. A pharmaceutical composition for preventing or treating coronavirus disease 2019 (COVID-19), comprising:
a coronavirus antigen and a vaccine adjuvant including a lipopeptide and poly(I:C),
wherein the coronavirus antigen is one or more antigens selected from the group consisting of spike protein 1 (S1), receptor-binding domain (RBD) and nucleoprotein (NP) of SARS-CoV-2 virus,
wherein the lipopeptide is at least one selected from the group consisting of Pam3Cys-SKKKK, PHC-SKKKK, Ole2PamCys-SKKKK, Pam2Cys-SKKKK, PamCys(Pam)-SKKKK, Ole2Cys-SKKKK, Myr2Cys-SKKKK, PamDhc-SKKKK, PamCSKKKK and Dhc-SKKKK, and
wherein the lipopeptide and poly(I:C) are in a weight ratio of 1 to 2:1.

11. A method for preventing, ameliorating or treating coronavirus disease 2019 (COVID-19), comprising:
a step of administering a pharmaceutical composition containing a coronavirus antigen and a vaccine adjuvant including a lipopeptide and poly(I:C) to a subject,
wherein the coronavirus antigen is one or more antigens selected from the group consisting of spike protein 1 (S1), receptor-binding domain (RBD) and nucleoprotein (NP) of SARS-CoV-2 virus,
wherein the lipopeptide is at least one selected from the group consisting of Pam3Cys-SKKKK, PHC-SKKKK, Ole2PamCys-SKKKK, Pam2Cys-SKKKK, PamCys(Pam)-SKKKK, Ole2Cys-SKKKK, Myr2Cys-SKKKK, PamDhc-SKKKK, PamCSKKKK and Dhc-SKKKK, and
wherein the lipopeptide and poly(I:C) are in a weight ratio of 1 to 2:1.

12.-13. (canceled)
